(19) United States
(12) Patent Application Publication
(10) Pub. No.: US 2026/0232216 A1
(43) Pub. Date: Aug. 13, 2026

(54) METHOD FOR IMPLEMENTING 3D REPRESENTATION OF BILIARY TRACT USING AI AND APPARATUS THEREFOR

(71) Applicant: SAMSUNG LIFE PUBLIC WELFARE FOUNDATION, Seoul (KR)

(72) Inventors: Jin Soo Rhu, Seoul (KR); Jae Won Joh, Seoul (KR); Gyu Seong Choi, Seoul (KR); Jong Man Kim, Seoul (KR); Woo Kyoung Jeong, Seoul (KR); Jae Hun Kim, Seoul (KR); So Young Lim, Seoul (KR)

(21) Appl. No.: 19/155,154

(22) PCT Filed: Dec. 14, 2023
(86) PCT No.: PCT/KR2023/020687

§ 371 Date: Aug. 8, 2025

(30) Foreign Application Priority Data
Feb. 28, 2023 (KR) 10-2023-0027259

(51) Int. Cl. A61B 5/055; A61B 5/00; G06T 17/00

(52) U.S. Cl. CPC A61B 5/055; A61B 5/004; A61B 5/7267; G06T 17/00; A61B 2576/02; G06T 2210/36; G06T 2210/41

(57) ABSTRACT

The present invention relates to a method for implementing a three-dimensional (3D) representation of the biliary tract using AI and an apparatus therefor. A method for creating a 3D representation of the biliary tract using AI according to an embodiment of the present invention may include the steps of: reconstructing abdominal image data including the biliary tract captured by magnetic resonance cholangiopancreatography; inputting the reconstructed image data into trained artificial intelligence (AI); identifying the biliary tract from the reconstructed abdominal image data using the trained AI; and converting the identified biliary tract into a 3D representation, wherein the trained AI may identify and output the biliary tract from the input abdominal image data including the biliary tract.

### TECHNICAL FIELD

The present invention relates to a method for implementing a three-dimensional (3D) representation of the biliary tract using AI and an apparatus therefor, and more particularly, to a method and apparatus for creating a 3D representation of the biliary tract using AI based on images captured by an imaging device.

### BACKGROUND ART

The biliary tract (or bile duct) refers to the pathway through which bile, a secretion of hepatocytes, is transported. The bile canaliculi, which are interconnected in a mesh-like structure, connect to interlobular bile ducts at the periphery of hepatic lobules. These interlobular bile ducts converge from the left and right lobes to form the common bile duct, which exits the liver through the hepatic hilum via the left and right hepatic ducts.

Although the biliary tract is small in size, its structure is long and complex, making it difficult to accurately identify the structure of the biliary tract using computed tomography (CT) or tomographic imaging, and thus cholangiography is used. Types of cholangiography include magnetic resonance cholangiopancreatography (MRCP), endoscopic retrograde cholangiopancreatography (ERCP), and percutaneous transhepatic cholangiography (PTC). Magnetic resonance cholangiopancreatography is a diagnostic method for acquiring images of the pancreaticobiliary system using a magnetic resonance imaging (MRI) device without the use of contrast media. Endoscopic retrograde cholangiopancreatography (ERCP) is a diagnostic method in which a contrast medium is injected into the biliary tract and the pancreatic duct through the duodenal papilla using a duodenoscope and a fluoroscope, thereby obtaining images of the biliary and pancreatic ducts, unlike general gastroscopy or colonoscopy. Percutaneous transhepatic cholangiography (PTC) is a diagnostic method in which a thin puncture needle is percutaneously inserted into the liver to access the intrahepatic bile ducts, and a contrast medium is injected to obtain images of the gallbladder and biliary tract.

### DETAILED DESCRIPTION OF THE INVENTION

### Technical Problem

Magnetic resonance cholangiopancreatography is based on 2D image data of the biliary tract captured by a magnetic resonance imaging (MRI) device. To convert the 2D image data of the biliary tract into a 3D representation (or 3D image), additional processing by a specialist is required. Although the 3D-converted images are highly useful for clinicians, it is difficult in terms of time and cost to request a specialist to perform additional processing each time to convert the 2D image data of the biliary tract into 3D.

Accordingly, the present invention proposes a method for implementing a 3D representation of the biliary tract using AI and an apparatus therefor.

### Technical Solution

A method for creating a three-dimensional (3D) representation of the biliary tract using artificial intelligence (AI) according to an embodiment of the present invention may include the steps of: reconstructing abdominal image data including the biliary tract captured by magnetic resonance cholangiopancreatography; inputting the reconstructed image data into trained artificial intelligence (AI); identifying the biliary tract from the reconstructed abdominal image data using the trained AI; and converting the identified biliary tract into a 3D representation, wherein the trained AI may identify and output the biliary tract from the input abdominal image data including the biliary tract.

In the method for creating a 3D representation of the biliary tract according to an embodiment of the present invention, the abdominal image data including the biliary tract captured by the magnetic resonance cholangiopancreatography may be MRCP GRASE abdominal image data.

In the method for creating a 3D representation of the biliary tract according to an embodiment of the present invention, the trained AI may be a U-Net model based on a deep learning model.

In the method for creating a 3D representation of the biliary tract according to an embodiment of the present invention, the step of reconstructing abdominal image data including the biliary tract captured by magnetic resonance cholangiopancreatography may include the steps of: reconstructing the voxel resolution of the image data to 1.0×1.0×1.0 mm; clipping the arbitrary unit (AU) values of the reconstructed image data within a predetermined range; and converting the AU values of the reconstructed image data into values between 0 and 1.

A computer-readable recording medium for creating a 3D representation of the biliary tract according to the present invention may record a computer program for executing at least one of the methods described above.

An electronic apparatus for creating a 3D representation of the biliary tract according to the present invention may execute at least one of the methods described above.

### Advantageous Effects

The present invention may assist in the diagnosis and treatment of diseases related to the biliary tract.

According to the present invention, it is possible to convert 2D abdominal image data captured by magnetic resonance cholangiopancreatography into a 3D representation without the assistance of a specialist.

According to the present invention, it is possible to enhance the anatomical understanding of a surgeon performing a surgery related to the biliary tract.

According to the present invention, a surgeon can easily explain biliary tract-related diseases to a patient, and the patient can readily understand such diseases, thereby facilitating effective communication between the surgeon and the patient.

### BRIEF DESCRIPTION OF DRAWINGS

FIG. 1 is a diagram illustrating the biliary tract and its surrounding organs.

FIGS. 2A to 2F illustrate examples of MRCP GRASE abdominal image data captured by magnetic resonance cholangiopancreatography from various directions for use in AI training, and corresponding image data in which the biliary tract is masked in the MRCP GRASE image data.

FIGS. 3A to 3C illustrate examples of binary mask images of the biliary tract and gallbladder for use in AI training.

FIGS. 4A to 4D illustrate examples of the biliary tract and gallbladder converted into 3D representations and viewed from various directions.

FIGS. 5A and 5B illustrate examples of the pancreaticobiliary system converted into 3D representations from MRCP GRASE image data captured by magnetic resonance cholangiopancreatography by a specialist and by AI, respectively.

FIG. 6 is a flowchart of a method for creating a 3D representation of the biliary tract using trained AI according to an embodiment of the present invention.

FIG. 7 is a block diagram of an electronic apparatus for creating a 3D representation of the biliary tract according to an embodiment of the present invention.

### MODE FOR CARRYING OUT THE INVENTION

The following embodiments are provided solely for the purpose of more specifically describing the present invention, and it will be apparent to those skilled in the art that the scope of the present invention is not limited by these embodiments in accordance with the gist of the invention. It should be understood that all modifications, equivalents, and substitutions within the spirit and scope of the present invention as described below are included.

It should be understood that the singular forms used herein include the plural forms unless the context clearly dictates otherwise. Moreover, it should be understood that terms such as “comprise” and the like indicate the presence of the described features, numbers, steps, operations, elements, or components, or combinations thereof, but do not preclude the presence or addition of one or more other features, numbers, steps, operations, elements, components, or combinations thereof. The term “and/or” includes any and all combinations of the associated listed items, or any one of the associated listed items.

In performing a method or an operation method, the respective processes or steps constituting the method may occur in an order different from the stated order, unless the context clearly states a specific order. That is, the respective processes or steps may occur in the stated order, may be performed substantially simultaneously, or may be performed in the reverse order.

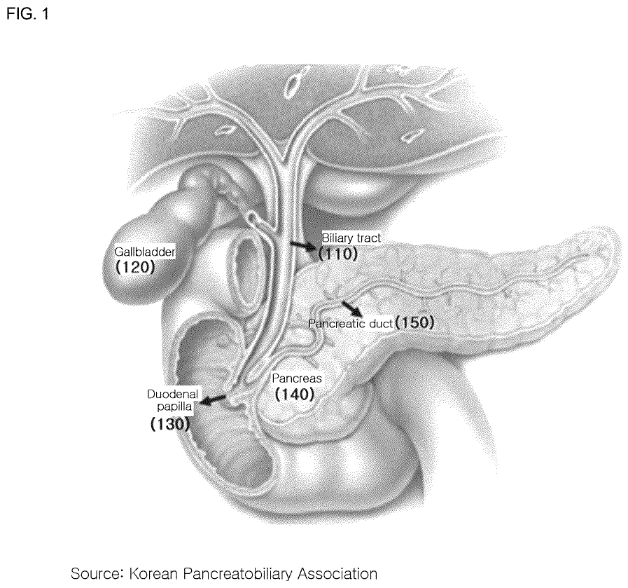

FIG. 1 is a diagram illustrating the biliary tract and its surrounding organs.

Referring to FIG. 1, organs surrounding the biliary tract (or, bile duct) 110 include the liver, the gallbladder 120, the duodenum, and the pancreas 140. The biliary tract 110 is a passage through which bile produced the liver is transported to the gallbladder 120 and the duodenum. More specifically, the portion of the biliary tract located in the liver is referred to as the intrahepatic biliary tract, and the portion extending from the outside of the liver to the duodenum is referred to as the extrahepatic biliary tract. The gallbladder 120 is located on the underside of the right lobe of the liver and stores bile. Bile transported through the biliary tract 110 may be secreted into the duodenum through the duodenal papilla 130.

Hereinafter, a method for implementing (or creating) a 3D representation of the biliary tract using trained AI and an apparatus therefor will be described in detail.

### Training Method for Utilizing Artificial Intelligence (AI)

The dataset (including input data and output data) to be used for AI training may be composed of MRCP GRASE (gradient and spin echo) image data of the abdomen including the biliary tract, captured by magnetic resonance cholangiopancreatography, and corresponding image data in which the biliary tract is indicated in the image data. GRASE image data refers to image data acquired using a GRASE sequence, which is one of the fast MRI techniques capable of rapidly acquiring high-resolution images. The image data acquired using the GRASE sequence may be composed of multiple gradient echo data obtained between 180-degree radio frequency pulses by combining the gradient echo method and the spin echo method. MRCP source images without contrast media can be acquired in a single breath-hold using the GRASE sequence, thereby enabling the reconstruction of high-precision 3D images of the biliary tract. In the GRASE image data, the biliary tract and its surrounding tissues are clearly distinguishable. In particular, the anatomical structures in GRASE image data can be aligned, as their positions are continuous across the superior-inferior direction of each slice, which makes the image data suitable for implementing a 3D structure. That is, the GRASE image data may be optimized for 3D implementation by minimizing errors due to breathing. In the present invention, the MRCP GRASE image data of the abdomen captured by magnetic resonance cholangiopancreatography serves as input data, and the image data in which the biliary tract is indicated within the MRCP GRASE image data serves as output data. To indicate the biliary tract in the MRCP GRASE image data, a semi-automated function of a medical imaging program may first be used, followed by manual processing by a specialist. The medical imaging program may include, for example, at least one of Mimics Medical by Materialise, Medip by MEDICALIP, Aview by Coreline Soft, and open-source programs such as 3D Slicer or inVesalius, but is not limited thereto.

In the present invention, by way of example, approximately 250 cases of MRCP GRASE image data and corresponding image data in which the biliary tract (or pancreaticobiliary system) is masked within the image data were used as the AI training dataset.

The MRCP GRASE image data input for AI training may be reconstructed as follows. For example, the voxel resolution of the input image data may be reconstructed to 1.0×1.0×1.0 mm, and the arbitrary unit (AU) values of the reconstructed image data may be clipped within a predetermined range and then converted into values between 0 and 1. Here, the AU is a relative measurement unit that represents the ratio of a measured value to a predetermined reference measurement, and the predetermined range may be −300 to 300.

AI training may be performed using, for example, a deep learning model. The deep learning model may be a U-Net model that utilizes segmented image data. In the U-Net model, the encoder blocks may be configured with ResNet-based modules, and the decoder blocks may be configured with ConvNet-based modules. The biliary tract may be indicated and output in the final layer.

To increase the amount of data used for AI training, at least one of flipping, rotation, and scaling may be performed on the preprocessed original image data.

Subsequently, deep learning may be performed using Adam optimization and Dice loss.

After performing training more than 1000 times using the training data, the weights of the deep learning model at that time may be determined and stored as the weights of the deep learning model. 90% of the total data may be used as training data, and the remaining 10% may be used for validating the deep learning model.

In the present invention, the training results obtained using deep learning were analyzed and verified by evaluating the success of the implementation according to the location of the bile duct. When compared with the results implemented by a specialist, a score of 1 was assigned if the representation of the bile duct was fully implemented by AI, 0.5 if it was partially implemented with some missing segments, and 0 if the implementation failed. Based on a total score of 100, the results achieved by AI were as follows: 92 for the common bile duct, 96 for the common hepatic duct, 70 for the cystic duct, 99 for the gallbladder, 96 for the hilar bile duct, 100 for the right hepatic duct, 86 for the right anterior hepatic duct, 72 for the right posterior hepatic duct, and 98 for the left hepatic duct. Overall, the scores were relatively high.

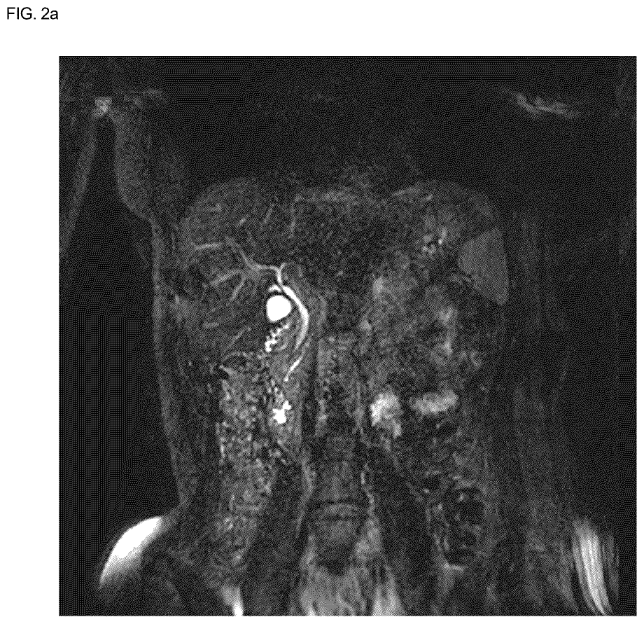

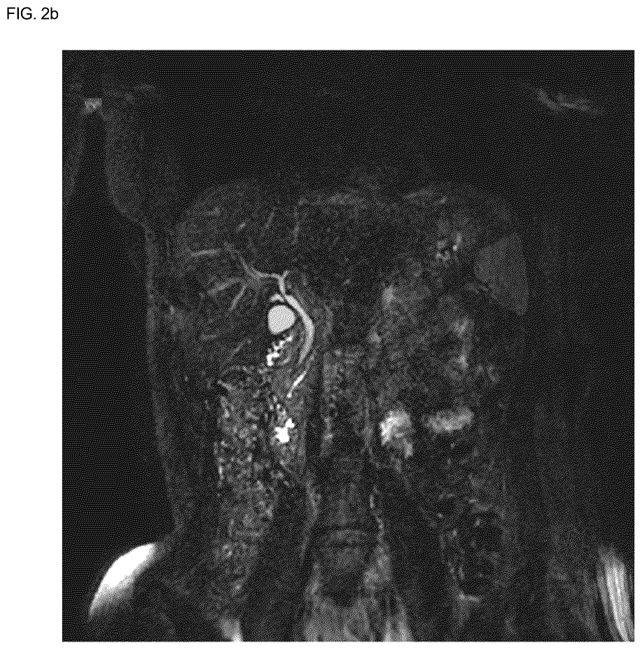

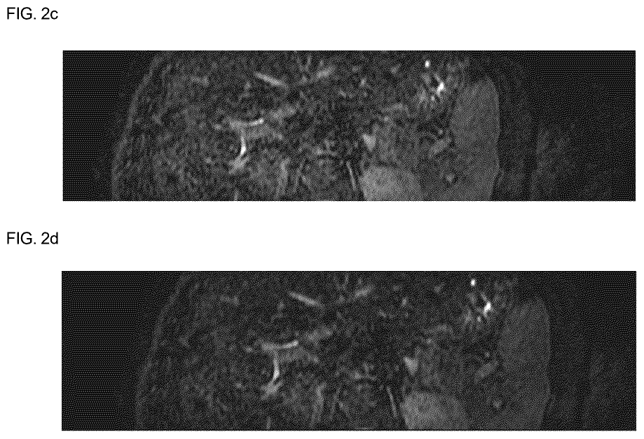

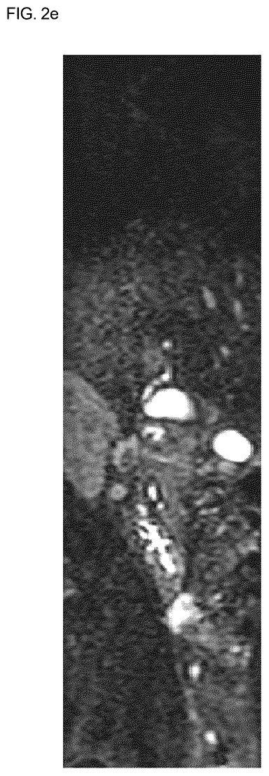

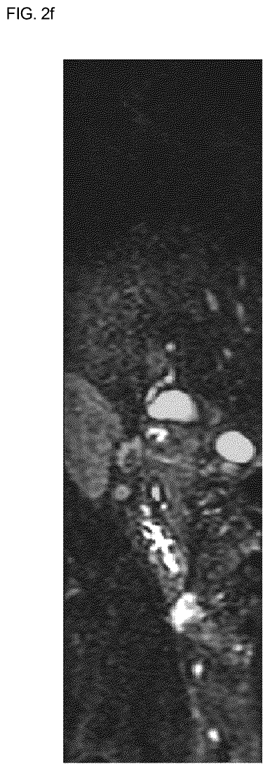

FIGS. 2A to 2F illustrate examples of MRCP GRASE abdominal image data captured by magnetic resonance cholangiopancreatography from various directions for use in AI training, and corresponding image data in which the biliary tract is masked in the MRCP GRASE image data.

Specifically, FIG. 2A shows MRCP GRASE abdominal image data, captured by magnetic resonance cholangiopancreatography and viewed from the coronal plane, and FIG. 2B shows the corresponding image data in which the biliary tract is masked in FIG. 2A. FIG. 2C shows MRCP GRASE abdominal image data, captured by magnetic resonance cholangiopancreatography and viewed from the axial plane, FIG. 2D shows the corresponding image data in which the biliary tract is masked in FIG. 2C. FIG. 2E shows MRCP GRASE abdominal image data captured by magnetic resonance cholangiopancreatography and viewed from the sagittal plane, and FIG. 2F shows the corresponding image data in which the biliary tract is masked in FIG. 2E. In other words, FIGS. 2B, 2D, and 2F may represent image data in which the biliary tract was manually masked by a specialist based on the MRCP GRASE image data shown in FIGS. 2A, 2C, and 2E, respectively. These masked image data, as shown in FIGS. 2B, 2D, and 2F, may be used to create a 3D representation of the biliary tract.

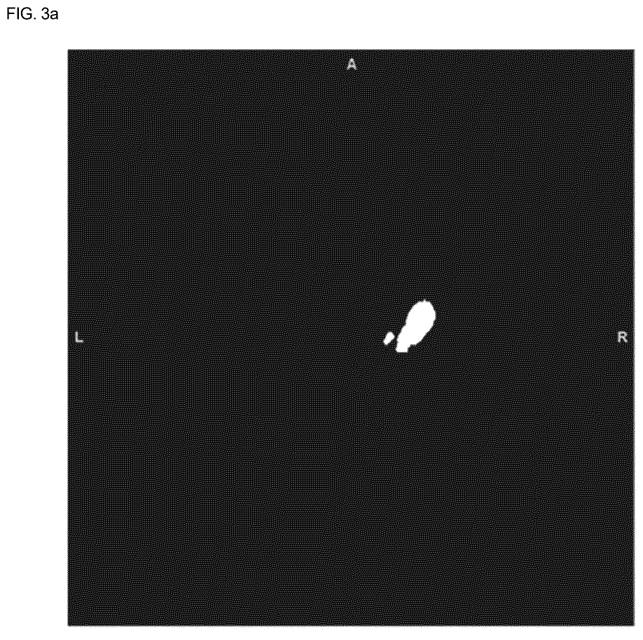

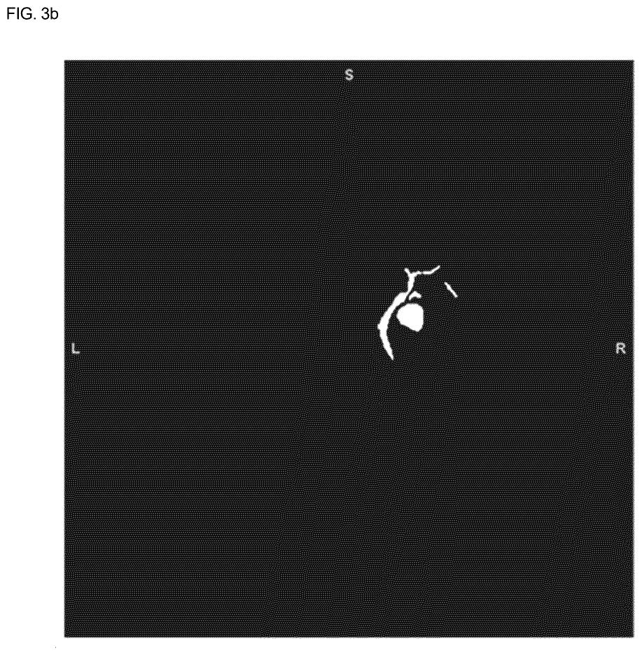

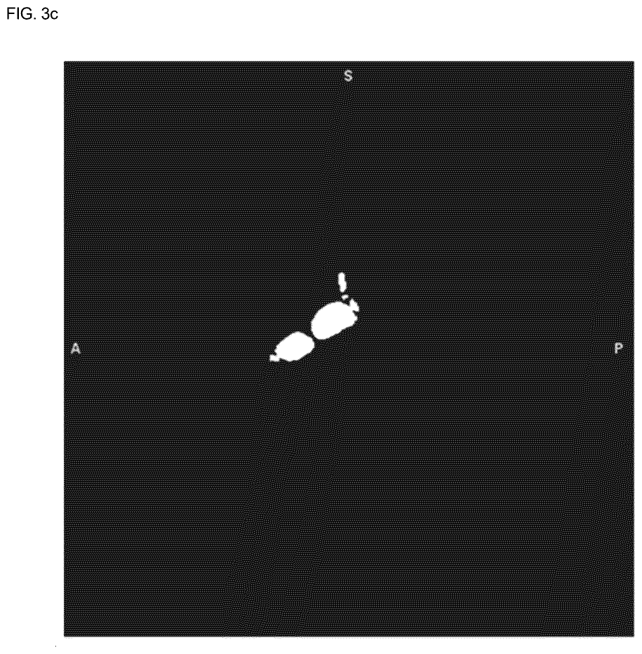

FIGS. 3A to 3C illustrate examples of binary mask images of the biliary tract and gallbladder for use in AI training.

Specifically, FIG. 3A shows a binary mask image indicating the biliary tract and gallbladder based on MRCP GRASE image data captured in the axial plane by magnetic resonance cholangiopancreatography, and FIG. 3B shows a binary mask image indicating the biliary tract and gallbladder based on MRCP GRASE image data captured in the coronal plane by magnetic resonance cholangiopancreatography. FIG. 3C shows a binary mask image indicating the biliary tract and gallbladder based on MRCP GRASE image data captured in the sagittal plane by magnetic resonance cholangiopancreatography. Here, the binary mask image refers to an image that indicates only the biliary tract and gallbladder, where the biliary tract and gallbladder regions are indicated either as 0 and all other regions as 1, or the biliary tract and gallbladder regions are indicated either as 1 and all other regions as 0. FIGS. 3A to 3C can be used as output data in the dataset for AI training.

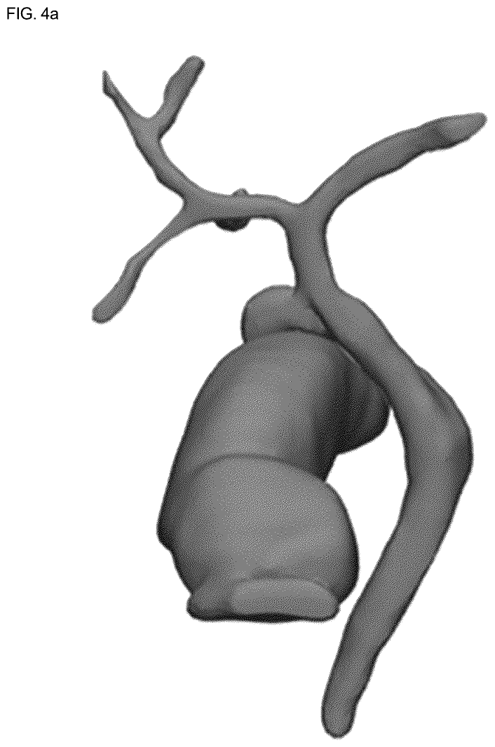

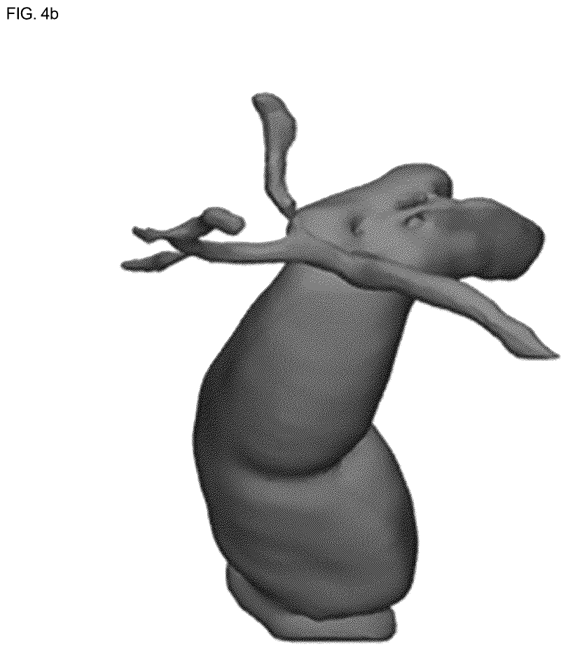

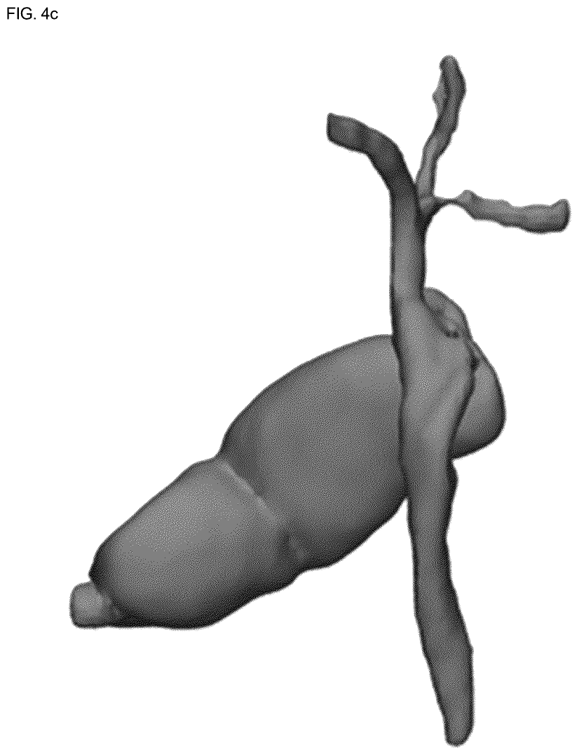

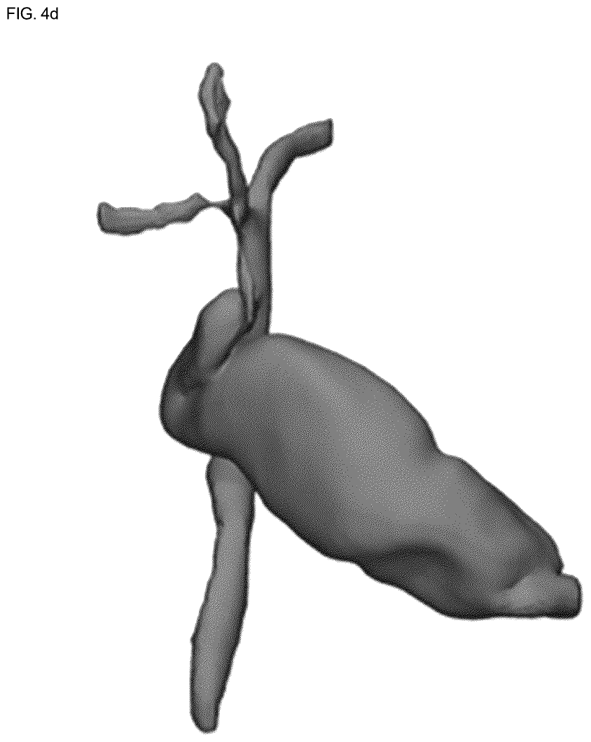

FIGS. 4A to 4D illustrate examples of the biliary tract and gallbladder converted into 3D representations and viewed from various directions.

Specifically, FIG. 4A illustrates an example of a front view of the biliary tract and gallbladder converted into a 3D representation, and FIG. 4B illustrates an example of a top view of the biliary tract and gallbladder converted into a 3D representation. FIG. 4C illustrates an example of a right view of the biliary tract and gallbladder converted into a 3D representation, and FIG. 4D illustrates an example of a left view of the biliary tract and gallbladder converted into a 3D representation. According to an embodiment, a user may control the 3D-converted biliary tract and gallbladder images to view them from various directions.

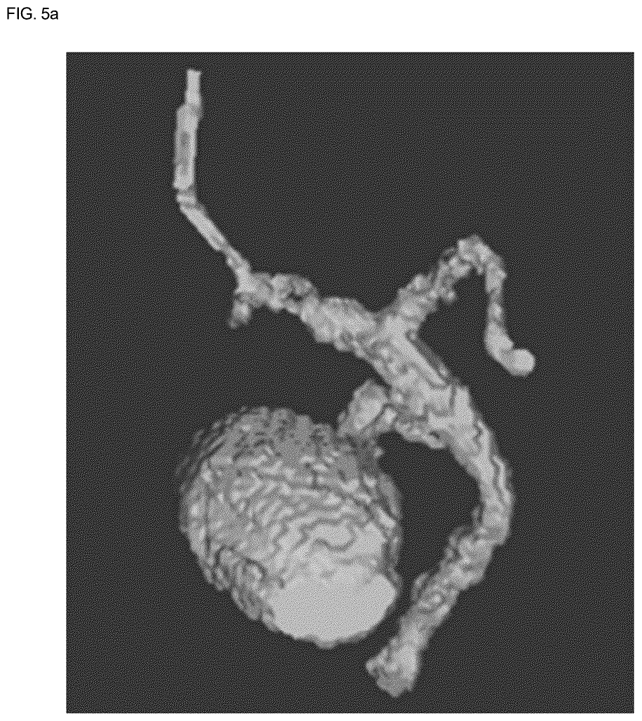

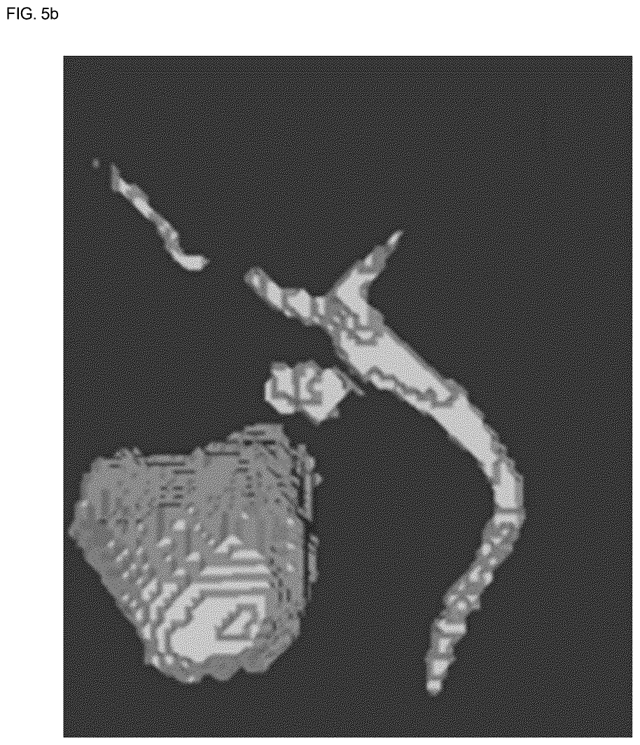

FIGS. 5A and 5B illustrate examples of the biliary tract and gallbladder converted into 3D representations from MRCP GRASE image data captured by magnetic resonance cholangiopancreatography by a specialist and by AI, respectively.

The specialist and AI may implement a 3D representation of the biliary tract and gallbladder from MRCP GRASE image data captured by magnetic resonance cholangiopancreatography. Specifically, FIG. 5A illustrates an example of the biliary tract and gallbladder implemented in 3D by a specialist from 2D MRCP GRASE image data captured by magnetic resonance cholangiopancreatography, by specialist, and FIG. 5B illustrates an example of the biliary tract and gallbladder implemented in 3D by AI from the same MRCP GRASE image data used in FIG. 5A.

The comparison between FIGS. 5A and 5B shows that the shape and size of the biliary tract and gallbladder are similar.

### Method for Creating a 3D Representation of the Biliary Tract Using Trained AI

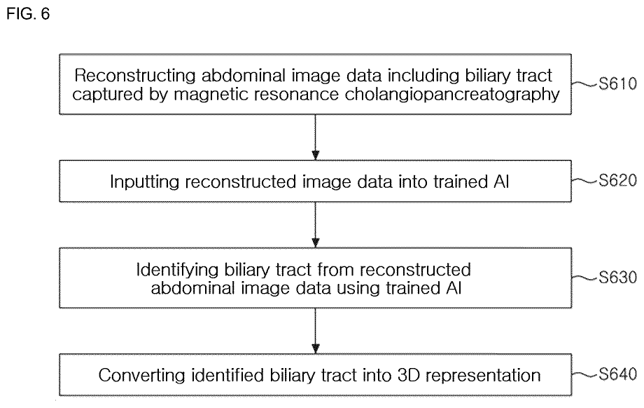

FIG. 6 is a flowchart of a method for creating a 3D representation of the biliary tract using trained AI according to an embodiment of the present invention.

Referring to FIG. 6, the method for creating a 3D representation of the biliary tract according to an embodiment of the present invention may include the step of reconstructing abdominal image data including the biliary tract captured by magnetic resonance cholangiopancreatography (S610). The abdominal image data including the biliary tract captured by the magnetic resonance cholangiopancreatography may be MRCP GRASE abdominal image data. Magnetic resonance cholangiopancreatography is a method for obtaining images of the biliary tract using a magnetic resonance imaging device without the use of contrast media.

According to an embodiment of the present invention, the step of reconstructing abdominal image data including the biliary tract captured by magnetic resonance cholangiopancreatography may include the steps of: reconstructing the voxel resolution of the image data to 1.0×1.0×1.0 mm; clipping the AU values of the reconstructed image data within the range of −300 to 300; and converting the AU values of the reconstructed image data into values between 0 and 1.

According to an embodiment of the present invention, the method for creating a 3D representation of the biliary tract may include the step of inputting the reconstructed image data into trained AI (S620). The trained AI may be an AI trained to identify and output the biliary tract when abdominal image data including the biliary tract is input. According to an embodiment, the AI may be a U-Net model based on a deep learning model.

According to an embodiment of the present invention, the method for creating a 3D representation of the biliary tract may include the step of identifying the biliary tract from the reconstructed abdominal image data using the trained AI (S630).

Finally, the method for creating a 3D representation of the biliary tract may include the step of converting the identified biliary tract into a 3D representation (S640).

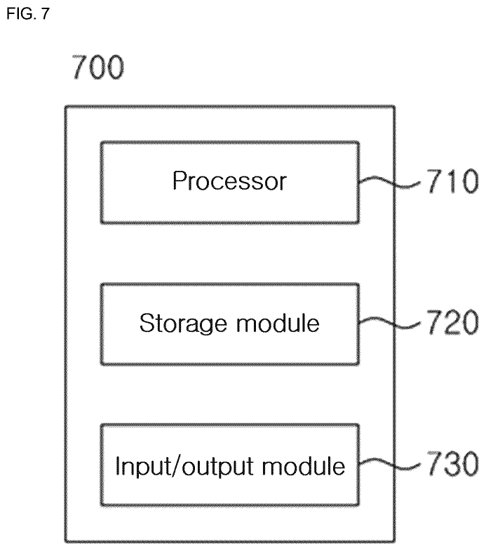

FIG. 7 is a block diagram of an electronic apparatus for creating a 3D representation of the biliary tract according to an embodiment of the present invention.

The electronic apparatus 700 may include a processor 710, a storage module 720, and an input/output module 730. The electronic apparatus 700 may further include a communication module (not shown).

The processor 710 may reconstruct abdominal image data including the biliary tract captured by magnetic resonance cholangiopancreatography. The abdominal image data including the biliary tract captured by magnetic resonance cholangiopancreatography may be MRCP GRASE abdominal image data. The magnetic resonance cholangiopancreatography may obtain images of the biliary tract using a magnetic resonance imaging device without the use of contrast media. The processor 710 may input the reconstructed image data into the trained AI. The trained AI may be an AI trained to identify and output the biliary tract when abdominal image data including the biliary tract is input. The processor 710 may identify the biliary tract from the reconstructed abdominal image data using the trained AI. The processor 710 may convert the identified biliary tract into a 3D representation.

The storage module 720 may store the trained AI. According to an embodiment, the trained AI may be a U-Net model based on a deep learning model. The trained AI may identify and output the biliary tract from the input abdominal image data including the biliary tract. According to an embodiment, the trained AI may be stored on an external server, in which case data input and output may be performed via the communication module.

The input/output module 730 may receive abdominal image data including the biliary tract captured by magnetic resonance cholangiopancreatography and may output the biliary tract created in 3D. Although the input/output module 730 is illustrated as a single module here, it may be configured as separate modules. For example, the input module may be a mouse and/or a keyboard, and the output module may be a display module such as a monitor or a speaker.

FIGS. 6 and 7 illustrate the method and apparatus for implementing a 3D representation of the biliary tract; however, according to an embodiment of the present invention, the gallbladder, in addition to the biliary tract, may also be implemented in 3D.

Although certain embodiments of the present invention have been described in detail above, it will be apparent to those skilled in the art that such detailed descriptions are merely exemplary of preferred embodiments and should not be construed as limiting the scope of the present invention. Thus, the substantial scope of the present invention will be defined by the appended claims and equivalents thereof.

1. A method for creating a three-dimensional (3D) representation of the biliary tract, the method comprising the steps of:
reconstructing abdominal image data including the biliary tract captured by magnetic resonance cholangiopancreatography;
inputting the reconstructed image data into trained artificial intelligence (AI);
identifying the biliary tract from the reconstructed abdominal image data using the trained AI; and
converting the identified biliary tract into a 3D representation,
wherein the trained AI identifies and outputs the biliary tract from the input abdominal image data including the biliary tract.

2. The method for creating a three-dimensional (3D) representation of the biliary tract according to claim 1, wherein the abdominal image data including the biliary tract captured by the magnetic resonance cholangiopancreatography is MRCP GRASE (gradient and spin echo) abdominal image data.

3. The method for creating a three-dimensional (3D) representation of the biliary tract according to claim 1, wherein the trained AI is a U-Net model based on a deep learning model.

4. The method for creating a three-dimensional (3D) representation of the biliary tract according to claim 1, wherein the step of reconstructing abdominal image data including the biliary tract captured by magnetic resonance cholangiopancreatography comprises the steps of:
reconstructing the voxel resolution of the image data to 1.0×1.0×1.0 mm;
clipping the arbitrary unit (AU) values of the reconstructed image data within a predetermined range; and
converting the AU values of the reconstructed image data into values between 0 and 1.

5. A computer-readable recording medium recording a computer program for executing the method according to claim 1.

6. An electronic apparatus for executing the method according to claim 1.
